(19) United States
(12) Plant Patent Application Publication
(10) Pub. No.: US 2005/0223450 P1
(43) Pub. Date: Oct. 6, 2005

(54) CALIBRACHOA PLANT NAMED ' SUNBELPAPI'

(50) PETUNIA HYBRIDA; PETUNIA

(76) Inventor: Takeshi Kanaya, Omihachiman (JP)

Correspondence Address: CHRISTIE, PARKER & HALE, LLP, PO BOX 7068, PASADENA, CA, 91109-7068, US

(21) Appl. No.: 10/814,614

(22) Filed: Mar. 30, 2004

(51) Int. Cl. A01H 5/00

(52) U.S. Cl. PLT/263

(57) ABSTRACT

Disclosed herein is a new and distinct variety of Petunia plant having an outwardly spreading growth habit. The Petunia plant has abundant branching and a great profusion of blooms, the whole plant remaining in bloom for a considerable period of time. The flowers are single and small, the petals having strong purplish pink. The inside color of the corolla throat is vivid yellow and the outside of the corolla tube is light greenish yellow with purplish pink veins. The plant exhibits high resistance to heat, cold, rain and disease.

### BOTANICAL/COMMERCIAL CLASSIFICATION

Petunia hybrida/Petunia Plant

### VARIETAL DENOMINATION

cv. Sunbelpapi

### BACKGROUND OF THE VARIETY

The present invention relates to a new and distinct variety of Petunia plant originated from crossing a Petunia hybrid variety called ‘9PP11’, as the female parent, and ‘9CPP8’, as the male parent.

The Petunia is a popular plant that is used for flower bedding and potting in the summer season. There are only a few Petunia bred, such as ‘Sunberubu’ (U.S. Plant Pat. No. 9,754), ‘Sunbelchipi’ (U.S. Plant Pat. No. 10,355) and ‘Sunbelki’ (U.S. Plant Pat. No. 11,558). These are of the spreading type, a medium plant height, abundant branches, and a high resistance to heat and rain and disease. However there are only a few varieties having a great profusion of flowers, having a strong purplish pink color, and a high resistance to rain, heat, and disease. Accordingly, this invention was aimed at obtaining a new Petunia variety having strong purplish pink and very small flowers, combined with the above features.

Progress

The female parent ‘9PP11’, (unpatented) used in the crossing of ‘Sunbelpapi’ is a strain of our breeding lines grown at Yokaichi-shi, Shiga-ken, Japan, having an outwardly spreading growth habit with abundant branching. It has small single flowers, the petals having a vivid reddish purple color.

The male parent ‘9CPP8’ (unpatented) used in the crossing of ‘Sunbelpapi’ is a strain of our breeding lines grown at Yokaichi-shi, Shiga-ken, Japan, having a decumbent growth habit with abundant branching. It has small single flowers, the petals having a vivid reddish purple color.

In April 1998, crossing of ‘9PP11’, as the female parent and ‘9CPP8’ as the male parent was conducted at Yokaichi-shi, Shiga-ken, Japan. In September 1998, 50 seedlings were obtained from that crossing. One seedling was selected in view of its growth habit, flower size and color in October 1999. That seedling was propagated by cutting, and the cuttings were grown in field and evaluated from April to September 2000. A trial was carried out by flower potting in the greenhouse and field from April to October 2001 at Yokaichi-shi, Shiga-ken, Japan. The botanical characteristics of that plant were then examined, using similar varieties ‘Sunbelchipi’ and ‘Sunbelkupapi’ for comparison. As a result, it was concluded that this Petunia plant is distinguishable from any other variety whose existence is known to us, and is uniform and stable in its characteristics. Then the new variety of Petunia plant was named ‘Sunbelpapi’.

In the following description, the color-coding is in accordance with the Horticultural Colour Chart of The Royal Horticultural Society, London, England (R.H.S. Colour Chart).

### SUMMARY OF THE VARIETY

This new variety is unlike any Petunia commercially available as evidenced by the following unique combinations of characteristics.

1. Compact and outwardly spreading growth habit with abundant branching.

2. Having a great profusion of blooms with the entire plant remaining in bloom for a considerable period of time.

3. The flowers are single and small. The petal color is strong purplish pink (R.H.S. 75A).

4. The plant has a high resistance to rain, cold, heat and disease.

The new variety ‘Sunbelpapi’ differs from the similar variety ‘Sunbelchipi’ in the following points.

1. The plant of ‘Sunbelpapi’ is more compact than that of ‘Sunbelchipi’.

2. The leaf of ‘Sunbelpapi’ is smaller than that of ‘Sunbelchipi’.

3. The leaf shape of ‘Sunbelpapi’ is elliptic. That of ‘Sunbelchipi’ is lanceolate.

4. The petal color of ‘Sunbelpapi’ is strong purplish pink (R.H.S. 75A). That of ‘Sunbelchipi’ is vivid purplish red (R.H.S. N57A).

5. The outside color of the corolla throat of ‘Sunbelpapi’ is light greenish yellow (R.H.S. 7D) with purplish pink (R.H.S. N74C) vein. That of ‘Sunbelchipi’ is brilliant greenish yellow (R.H.S. 5C).

6. The apex shape of petal of ‘Sunbelpapi’ is rounded. That of ‘Sunbelchipi’ is obtuse.

7. The peduncle length of ‘Sunbelpapi’ is longer than that of ‘Sunbelchipi’.

The new variety ‘Sunbelpapi’ differs from the similar variety ‘Sunbelkupapi’ in the following points.

1. The growth habit of ‘Sunbelpapi’ is outwardly spreading. That of ‘Sunbelkupapi’ is decumbent.

2. The plant height of ‘Sunbelpapi’ is higher than that of ‘Sunbelkupapi’.

3. The leaf of ‘Sunbelpapi’ is smaller than that of ‘Sunbelkupapi’.

4. The leaf shape of ‘Sunbelpapi’ is elliptic. That of ‘Sunbelkupapi’ is lanceolate.

5. The petal color of ‘Sunbelpapi’ is strong purplish pink (R.H.S. 75A). That of ‘Sunbelkupapi’ is deep purplish pink (R.H.S. 73A) with a yellowish white (R.H.S. 115C) eye color.

6. The peduncle length of ‘Sunbelpapi’ is longer than that of ‘Sunbelkupapi’.

This new variety of Petunia Plant ‘Sunbelkupapi’ was asexually reproduced by the use of cuttings at Omori-cho, Yokaichi-shi, Shiga-ken, Japan, and homogeneity and stability thereof were confirmed. The instant plant retains its distinctive characteristics and reproduces true to type in successive generations.

### BRIEF DESCRIPTION OF THE PHOTOGRAPH

The depicted plants had been reproduced by the use of cuttings and were photographed during September 2001 while growing outdoors in 24 cm pots, and at an age of approximately 8 months, at Yokaichi-shi, Shiga-ken, Japan.

FIG. 1 illustrates a typical plant of the new variety of Petunia plant ‘Sunbelpapi’.

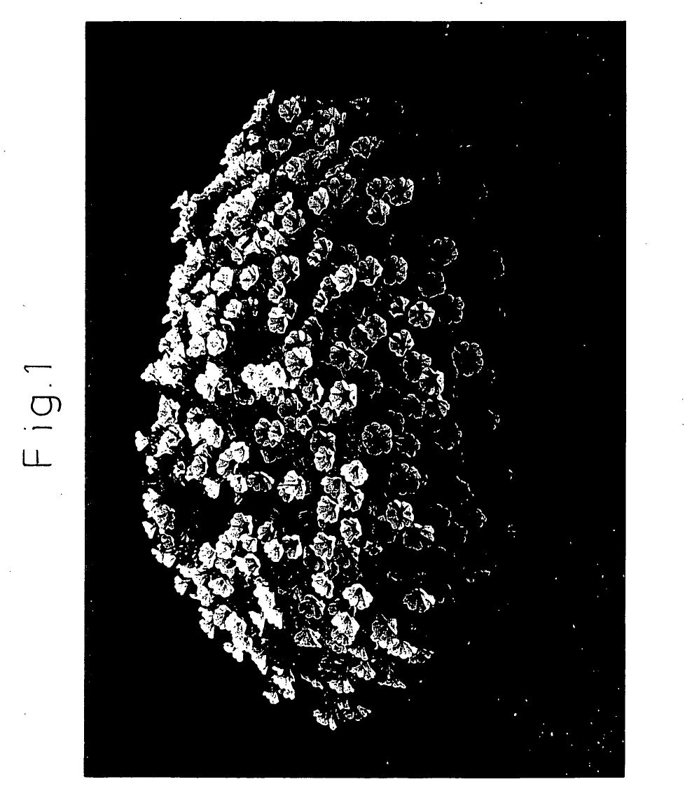

FIG. 2 illustrates a close-up view of typical foliage and blossoms of the new variety of Petunia plant ‘Sunbelpapi’.

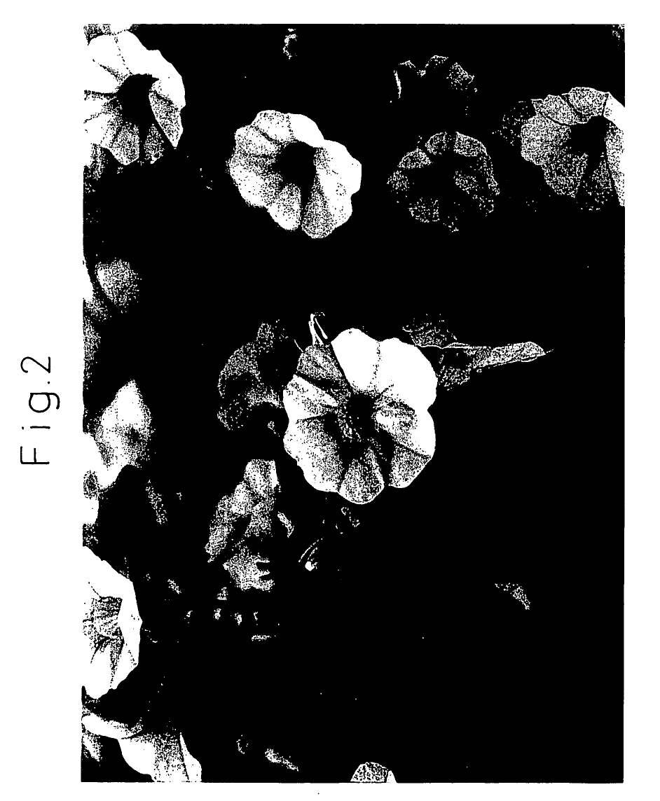

### DESCRIPTION OF THE VARIETY

The botanical characteristics of the new and distinct variety of Petunia plant named ‘Sunbelpapi’ are as follows, when observed during September at Yokaichi-shi, Shiga-ken, Japan at an age of approximately 9 months.
Plant:
   Growth habit.—Outwardly spreading.
   Plant height.—Approximately 14.6 cm.
   Spreading area of plant.—Approximately 45.6 cm.
   Blooming period.—April to late October in the southern Kanto area, Japan. The plant shape does not change throughout this period.
Stem:
   Thickness.—Approximately 1.3 mm.
   Pubescence.—Sparse.
   Branching.—Abundant branching, especially secondary branches.
   Internode length.—Approximately 0.6 cm.
   Color.—R.H.S. 144B, 200C (strong yellow green, grayish brown).
Leaf:
   Whole shape.—Elliptic. The apex shape is acute, and the base shape is attenuate.
   Length.—Approximately 1.9 cm.
   Width.—Approximately 0.8 cm.
   Color.—Upper side color is R.H.S. 146A (moderate olive green), bottom side color is R.H.S. 146B (moderate yellow green).
   Thickness.—Approximately 0.4 mm.
   Pubescence.—Sparse.
Flower:
   Facing direction.—Slanted upward.
   Type.—Single.
   Shape.—Funnel-shape, with five-fissures.
   Shape of petal tip.—Rounded.
   Lobation.—Shallow.
   Waving of petal.—Weak.
   Diameter.—Approximately 3.0 cm.
   Color.—Petal; R.H.S. 75A (strong purplish pink). Inside color of the corolla throat is R.H.S. 14B (vivid yellow). Outside color of the corolla throat is R.H.S. 7D (light greenish yellow) with R.H.S. N74C (purplish pink) vein.
   Reproductive organs.—1 normal pistil and 5 normal stamens. The stigma is club-shaped and R.H.S. N144D (light yellow green) in coloration. The style is approximately

4.7 mm in length and R.H.S. 145B (brilliant yellow green) in coloration. The ovary is R.H.S. N144D (light yellow green) in coloration. The stamens commonly are of variable length from approximately 4.7 to 7.4 mm. Pollen is formed in a quantity that is typical of Petunia and is near R.H.S. 21A (vivid orange yellow) in coloration.
Fertility.—Fertile, but self-incompatible.
Peduncle.—Approximately 0.6 mm in diameter and approximately 4.5 cm in length. The texture is smooth.
Calyx.—Narrow. 5 sepals fused at the base.
Seeds.—R.H.S. N186A (strong red) in coloration, approximately 0.6 mm in diameter, and generally spherical. The quantity is typical of Petunia.
Physiological and ecological characteristics: High resistance to cold, heat, rain and disease such as Powdery mildew. The resistance to heat and rain is very strong.

This new variety of Petunia plant is most suitable for flower bedding and potting, particularly in hanging pots or planters. Pinching of old blossoms will enhance the formation of new blossoms.

1. A new and distinct variety of Petunia plant named ‘Sunbelpapi’, substantially as herein illustrated and described.
